(19) United States
(12) Patent Application Publication
(10) Pub. No.: US 2003/0186825 A1
(43) Pub. Date: Oct. 2, 2003

(54) Contact lens wetting solution

(75) Inventors: Motohiro Mitani, Toda-shi (JP); Ryota Ando, Tsukuba-shi (JP); Hirofumi Irie, Ushiku-shi (JP); Ken Suzuki, Tsukuba-shi (JP); Nobuo Nakabayashi, Matsudo-shi (JP); Kazuhiko Ishihara, Tokyo (JP)

Correspondence Address: DARBY & DARBY P.C., 805 Third Avenue, New York, NY, 10022, US

(73) Assignee: Nobuo Nakabayashi

(21) Appl. No.: 10/372,014

(22) Filed: Feb. 21, 2003

Related U.S. Application Data
(63) Continuation of application No. PCT/JP01/07169, filed on Aug. 22, 2001.

(30) Foreign Application Priority Data
Aug. 22, 2000 (JP) 2000-251261

(51) Int. Cl. A61K 31/74; C11D 1/00

(52) U.S. Cl. 510/112

(57) ABSTRACT

Contact lens wetting solutions for reducing friction between the contact lens and ocular tissues, and maintaining such effect for a long time, are disclosed. The wetting solution is composed only of a particular ratio of a lubricant composed of copolymer (A) obtained by polymerizing a monomer composition comprising 2-(meth)acryloyloxyethyl phosphorylcholine and alkyl(meth)acrylate, a buffer, an inorganic chloride, a preservative, a chelating agent, and water. The copolymer (A) has a weight average molecular weight of 100,000 to 1,000,000, and satisfies the formula 80≦N×R≦240, provided that 4≦N≦18 and 10%≦R≦60%, wherein N stands for the carbon number of the alkyl group in monomer Y constituting copolymer (A), and R stands for a molar fraction of the unit derived from monomer Y with respect to the sum of the units derived from monomers X and Y.

### FIELD OF ART

The present invention relates to wetting solutions for contact lenses. More specifically, the present invention relates to contact lens wetting solutions for use upon lens insertion, containing a lubricant for reducing friction between the contact lens and ocular tissues, such as the cornea, palpebral conjunctiva, or bulbar conjunctiva.

### BACKGROUND ART

Contact lenses are generally categorized into non-water content and hydrogel contact lenses. The non-water content contact lenses, which are made of stabler materials than those of hydrogel lenses, include, for example, contact lenses principally made of methyl methacrylate, and rigid gas permeable contact lenses principally made of silyl-methacrylate or fluoro-methacrylate. The non-water content contact lenses, when worn, give feeling of something in the eye for its material hardness. Due to this discomfort, not a few people have difficulty in wearing the non-water content contact lenses.

The hydrogel contact lenses, which are hydrogel of polyhydroxyethylmethacrylate or other water-soluble polymers, give less feeling of something in the eye, and provide more comfortable wear compared to the non-water content contact lenses. However, even the hydrogel contact lenses tend to become uncomfortable with the lapse of time due to evaporation of moisture from the lenses, which is caused by reduced blink rate induced by increased frequency of work at VDT (Visual Display Terminal) or reduced humidity in indoor environment

Such feeling of something in the eye is believed to be attributed to friction between the lens material and the ocular mucosa or tunica conjunctiva. For reducing such friction, there are proposed methods of interposing between the lens material and the ocular mucosa or tunica conjunctiva a wetting solution containing a polymer compound as a lubricant. As a polymer compound for this purpose, dextran or arabinogalactan (JP-52-70015-A), a mixture of polysaccharides and polyvinyl alcohol (PVA) (JP-53-13588-A), perfluorocarbon (JP-58-219125-A), and a mixture of PVA and polyvinyl pyrrolidone and hydroxymethyl cellulose, methyl cellulose, or carboxymethyl cellulose (JP-61-69023-A) are proposed.

Such wetting solutions exhibit a lubricating effect right after instillation over the lens, but require frequent instillation due to poor endurance of the effect.

Further, the above-mentioned mixture of polysaccharides (cellulose derivatives) and PVA has molecules of which hydroxyl groups are firmly hydrogen-bonded together, preventing water molecules from intruding between the molecules. Thus when powders of the mixture are directly introduced into water, only the powder surface dissolves to form coagulated particles, which require extremely long time to dissolve. It is thus difficult to prepare a wetting solution in a liquid form.

As an example of use of a polymer having a phosphorylcholine group in the field of ophthalmic solutions, JP-7-166154-A proposes a contact lens solution for giving aniflouling and hydrophilic properties to contact lenses. This publication discloses a copolymer prepared by polymerizing 2-methacryloyloxyethyl phosphorylcholine and butyl methacrylate in a 1:1 weight ratio (the molar ratio of the unit derived from 2-methacryloyloxyethyl phosphorylcholine to the unit derived from butyl methacrylate in the copolymer is 3:7). An aqueous solution of this copolymer was prepared, in which a contact lens was soaked, and the contact angle between the soaked contact lens and water was measured. The result suggests that the copolymer may improve comfort of lens wear. JP-10-324634-A proposes an ophthalmic pharmaceutical composition for prevention and treatment of dry eye.

In these publications, however, the lubricating effect of the polymers having a phosphorylcholine group to reduce friction between the contact lens and ocular tissues, was not specifically demonstrated by actual measurement of the friction. Thus it cannot be said that the polymers having a phosphorylcholine group specifically enumerated in the publications, have sufficient lubricating properties. These publications also do not discuss the specific factors required for giving the lubricating effect to a copolymer of 2-(meth)acryloyloxyethyl phosphorylcholine and alkyl (meth) acrylate.

### DISCLOSURE OF THE INVENTION

It is an object of the present invention to provide a contact lens wetting solution which reduces discomfort upon lens insertion and during lens wear, and which provides such a discomfort-reducing effect for a prolonged period of time.

The inventors have made intensive studies for achieving the above objects, to find that specific copolymers obtained by polymerizing a monomer composition containing 2-(meth)acryloyloxyethyl phosphorylcholine and alkyl(meth)acrylate have a lubricating effect to reduce friction between the contact lens and ocular tissues, and excellent safety, thereby completing the present invention, in which copolymers the alkyl group in the alkyl(meth)acrylate has a specific carbon number, the molar fraction of the constituting unit of the copolymer derived from alkyl(meth)acrylate with respect to the sum of the constituting units of the copolymer derived from 2-(meth)acryloyloxyethyl phosphorylcholine and alkyl(meth)acrylate is at a specific percent, and the molecular weight of the copolymer is restricted.

According to the present invention, there is provided a contact lens wetting solution consisting of 0.05 to 5% w/v of a lubricant consisting of a copolymer (A) obtained by polymerizing a monomer composition comprising 2-(meth)acryloyloxyethyl phosphorylcholine represented by the formula (1) (sometimes referred to as monomer X hereinbelow) and alkyl(meth)acrylate represented by the formula (2) (sometimes referred to as monomer Y hereinbelow), 0.1 to 1.5% w/v of a buffer, 0.1 to 1.5% w/v of an inorganic chloride, 0.00001 to 0.1% w/v of a preservative, 0.0001 to 0.1% w/v of a chelating agent, and water, wherein said copolymer (A) has a weight average molecular weight of 100000 to 1000000, and satisfies the formula 80≦N×R≦240, provided that 4≦N≦18 and 10%≦R≦60%, wherein N stands for a carbon number of an alkyl group in monomer Y constituting copolymer (A), and R stands for a molar fraction of a unit derived from monomer Y with respect to the sum of units derived from monomers X and Y:

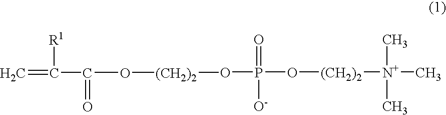

wherein R¹ stands for a hydrogen atom or a methyl group,

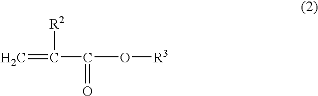

wherein R² stands for a hydrogen atom or a methyl group, and R³ stands for an alkyl group having 4 to 18 carbon atoms.

### PREFERRED EMBIDIMENTS OF THE INVENTION

The lubricant used in the contact lens wetting solution according to the present invention is made of a specific copolymer (A) which is obtained by polymerizing a monomer composition containing monomer X presented by the formula (1) and monomer Y represented by the formula (2), and has a weight average molecular weight of 100,000 to 1,000,000.

Monomer X is 2-methacryloyloxyethyl phosphorylcholine (referred to as MPC hereinbelow) or 2-acryloyloxyethyl phosphorylcholine, with the former being preferred for its ready availability.

Monomer X may be synthesized, for example, by reacting 2-hydroxyethyl(meth)acrylate and 2-chloro-2-oxo-1,3,2-dioxaphospholane in the presence of a dehydrochlorinating agent. Details of this reaction is disclosed in JP-11-43496-A, JP-58-154591-A, and Makromol.Chem (S.Nakai, T.Nakaya and M.Imoto;178., p2963,1977).

Monomer Y with R³ having not more than 3 carbon atoms hardly has a lubricating property, whereas that having not less than 19 carbon atoms has low solubility in a solvent used in preparation of the copolymer, thus being unusable.

Monomer Y may be, for example, a straight alkyl(meth)acrylate such as butyl(meth)acrylate, hexyl(meth)acrylate, octyl(meth)acrylate, lauryl(meth)acrylate, or stearyl(meth)acrylate; a branched alkyl(meth)acrylate such as isobutyl(meth)acrylate or 2-ethylhexyl(meth)acrylate; or a cyclic alkyl(meth)acrylate such as cyclohexyl(meth)acrylate. A single monomer Y or a mixture of two or more monomer Y's may be used in the polymerization.

In the monomer composition for preparation of copolymer (A), monomers X and Y contained therein must have a relationship satisfying the formula 80≦N×R≦240, provided that 4≦N≦18 and 10%≦R≦60%, wherein N stands for the carbon number of the alkyl group in monomer Y, and R stands for a molar fraction of the constituting unit of the objective copolymer A derived from monomer Y with respect to the sum of the constituting units derived from monomers X and Y. In other words, the blending ratio of monomers X and Y in the monomer composition must be decided depending on the carbon number of the alkyl group in monomer Y so that the above formula is satisfied.

In the monomer composition, for example the alkyl group in monomer Y has 4 carbon atoms, the contents of monomers X and Y must be such that the molar fraction R of the unit derived from monomer Y in copolymer (A) is 20 to 60%, while the molar fraction of the unit derived from monomer X is 40 to 80%, to satisfy the above formula. In another example wherein the alkyl group in monomer Y has 18 carbon atoms, the contents of monomers X and Y must be such that the molar fraction R of the unit derived from monomer Y in copolymer (A) is 10 to about 13.3%, while the molar fraction of the unit derived from monomer X is about 86.6 to 90%, to satisfy the above formula. When two or more kinds of monomer Y having the alkyl groups of different carbon numbers are used together as the monomer Y, for example when the monomer Y consists of 50 wt % of a monomer Y having 4 carbon atoms and 50 wt % of a monomer Y having 8 carbon atoms, the contents of monomers X and Y must be such that the molar fraction R of the unit derived from monomer Y is 10 to 30% for the unit derived from the monomer Y with 4 carbon atoms and 5 to 15% for the unit derived from the monomer Y with 8 carbon atoms, while the molar fraction of the unit derived from monomer X is 55 to 85%, to satisfy the above formula.

If the relationship between monomers X and Y does not satisfy the above formula, the lubricating property and the long-time endurance of such lubricating property required of the present invention are hard to be obtained.

According to the present invention, monomers in the monomer composition preferably consists only of monomers X and Y, but may include other copolymerizable monomers than X and Y, for improving the desired and other properties, as long as the desired effects of the present invention are not impaired. The content of such other copolymerizable monomers is preferably not more than 20 wt % of the monomer composition. In other words, the total content of monomers X and Y is preferably 80 to 100 wt % of the monomer composition.

Copolymer (A) may readily be prepared, usually by radical polymerization of the above monomer composition. For example, copolymer (A) may be prepared from the monomer composition by known radical polymerization, such as bulk polymerization, suspension polymerization, emulsion polymerization, or solution polymerization, in the presence of a polymerization initiator, while replacing the atmosphere with an inert gas such as nitrogen, carbon dioxide, or helium, or in an inert gas atmosphere. Among these, solution polymerization is preferred in view of the following purifying process.

The polymerization initiator may be any conventional radical polymerization initiators, for example, benzoyl peroxide, lauroyl peroxide, diisopropyl peroxydicarbonate, t-butylperoxy-2-ethylhexanoate, t-butylperoxypivalate, t-butylperoxydiisobutylate, azobisisobutyronitrile (abbreviated as AIBN hereinbelow), azobis-2,4-dimethylvaleronitrile, persulfate, or persulfate-hydrogensulfite.

The amount of the polymerization initiator is preferably 0.001 to 10 parts by weight, more preferably 0.01 to 5 parts by weight, based on 100 parts by weight of the monomer components of the monomer composition. The polymerization is performed preferably at 20 to 100° C., and preferably for 0.5 to 72 hours.

The molecular weight of the resulting copolymer (A), which may vary depending on the polymerization temperature and the amount of the polymerization initiator, polymerization modifier, or the like used in the polymerization, is 100,000 to 1,000,000 in weight average molecular weight. With a weight average molecular weight of less than 100,000, the lubricating property will not last for a sufficiently long time, whereas with more than 1,000,000, preparation is difficult.

The obtained copolymer (A) may be purified by an ordinary method, such as reprecipitation, dialysis, or ultrafiltration.

The obtained copolymer (A) has effects of mitigating physical discomfort upon lens insertion and during lens wear, specifically reducing friction between the contact lens and ocular tissues such as the cornea, palpebral conjunctiva, or bulbar conjunctiva, as well as maintaining such a mitigating effect for a prolonged period of time. The lubricant may be used as a component of contact lens wetting solutions or eye drops for use upon lens insertion, as will be discussed later.

The contact lens wetting solution according to the present invention is an aqueous solution which is to be contacted with a contact lens, for example, by instilling the solution over the lens prior to lens insertion, and which contains a specific ratio of the lubricant composed of copolymer (A), a buffer, an inorganic chloride, a preservative, and a chelating agent.

In the wetting solution of the present invention, the concentration of copolymer (A) as a lubricant is 0.05 to 5.0% weight per volume (% w/v), preferably 0.1 to 3.0% w/v. With the concentration of less than 0.05% w/v, the lubricating effect is insufficient, whereas with the concentration exceeding 5.0% w/v, the viscosity of the solution is too high.

The buffer contained in the present wetting solution functions to control the pH of the wetting solution. The buffer maybe, for example, hydrochloric acid, acetic acid, citric acid, sodium hydroxide, or boric acid; a borate such as borax; a phosphate such as monosodium phosphate, disodium phosphate, monopotassium phosphate, or dipotassium phosphate; a citrate such as sodium citrate; tris(hydroxymethyl)aminomethane; or mixtures thereof. Among various buffers, phosphates are most preferred.

The concentration of the buffer is 0.1 to 1.5% w/v, preferably 0.2 to 1.0% w/v. With the concentration of less than 0.1% w/v, the buffering capacity is too low to control the pH, whereas with the concentration exceeding 1.5% w/v, solubilities of other components are impaired.

The inorganic chloride contained in the present wetting solution functions to control permeability. The inorganic chloride may preferably be, for example, sodium chloride, potassium chloride, magnesium chloride, or mixtures thereof, with sodium chloride being the most preferred.

The concentration of the inorganic chloride is 0.1 to 1.5% w/v, preferably 0.2 to 1.0% w/v. With the concentration of less than 0.1% w/v or exceeding 1.5% w/v, deformation of contact lenses or eye irritation may occur.

The preservative contained in the present wetting solution functions as a disinfectant, and is not necessarily contained when, for example, unit dose containers are used, which are capable of preventing intrusion of bacteria, or when the wetting solution is for non-water content contact lenses. The preservative may be, for example, chlorhexidine gluconate, polyhexamethylene biguanide, benzalkonium chloride, paraben, or mixtures thereof, with chlorhexidine gluconate or polyhexamethylene biguanide being particularly preferred.

The concentration of the preservative 0.00001 to 0.1% w/v, preferably 0.0001 to 0.01% w/v. With the concentration exceeding 0.1% w/v, eye or skin irritation may occur or safety to cornea epithelial cells may be endangered.

The chelating agent contained in the present wetting solution functions to prevent calcium deposit on the contact lens by chelating. The chelating agent may be, for example, citric acid, ethylenediaminetetraacetic acid, cyclohexanediaminetetraacetic acid, an alkali metal salt thereof, or mixtures thereof, with disodium ethylenediaminetetraacetate being the most preferred.

The concentration of the chelating agent is 0.0001 to 0.1% w/v. With the concentration of less than 0.0001% w/v, the chelating capacity is not sufficient, whereas with the concentration exceeding 0.1% w/v, eye irritating property may be enhanced.

The wetting solution of the present invention is an aqueous solution consisting only of copolymer (A), the buffer, the organic chloride, the preservative, and the chelating agent.

The wetting solution of the present invention may readily be prepared by dissolving each of the above components in purified water at the ratio specified above. The purified water may be, for example, ion exchanged water, distilled water, water purified through reverse osmosis membrane, or ultrafiltered water.

The wetting solution of the present invention may be used by instilling the solution over the contact lens prior to lens insertion, and putting the lens on the eye to bring the solution into contact with ocular tissues.

Since the contact lens wetting solution of the present invention contains, at a specific ratio, the lubricant composed of copolymer (A), the buffer for controlling pH, the inorganic chloride for controlling permeability, the preservative acting as a disinfectant, and the chelating agent for chelating calcium, the wetting solution is capable of reducing friction between the contact lens and the cornea, palpebral conjunctiva, or bulbar conjunctiva, exhibiting a lubricating effect, and maintaining such an effect for a prolonged period of time.

### EXAMPLES

The present invention will now be explained in more detail with reference to Examples and Comparative Examples, which are illustrative only and do not intend to limit the present invention.

Procedures for measuring the weight average molecular weight of polymers in the following examples are discussed below.

Procedures for Measuring Weight Average Molecular Weight of Polymers

<Measurement of Molecular Weight>

An aqueous solution of a copolymer prepared in examples was diluted with a 20 mM phosphate buffer (pH 7.4) to 1.0% w/v, and passed through a 0.45 μm membrane filter, to prepare a test solution. The GPC analysis was conducted under the following conditions.

<Conditions for GPC Analysis>

Column: G3000PWXL and G6000PWXL arranged in series (manufactured by TOSOH CORPORATION); Eluent: 20 mM phosphate buffer (pH 7.4); Reference Material: polyethylene glycol (manufactured by POLYMER LABORATORIES LTD.); Detection: refractive index detector RI-8020 (manufactured by TOSOH CORPORATION); Calculation of Weight Average Molecular Weight (Mw), Number Average Molecular Weight (Mn) and Molecular Weight Distribution (Mw/Mn): molecular weight calculation program with integrator (GPC program for SC-8020) manufactured by TOSOH CORPORATION; Flow Rate: 0.5 mL/min.; Sample Solution Used: 10 μL; Column Temperature: 45° C.

<Determination of Compositional Ratio in Polymer>

5 mg of the polymer prepared in Synthesis Example was measured out, and the contents of elements C and N in the polymer were respectively measured with PerkinElmer 2400II CHNO/S Elemental Analyzer (manufactured by PERKINELMER INC.). The compositional ratio was calculated from the ratio of the amounts of elements C and N.

For example, when the polymer is made of MPC and n-butyl methacrylate (abbreviated as BMA), the compositional ratio of MPC and BMA is determined in accordance with the following formula, wherein the amount of the MPC component is put as x mol, and the BMA component as (1−x) mol:

Amount of Element N/Amount of Element C=Atomic Weight of Nitrogen 14.01×x/Atomic Weight of Carbon 12.01×Carbon Number 11×x+Atomic Weight of Carbon 12.01×Carbon Number 8×(1−x)

### Synthesis Example 1-1

45 g of MPC, 5 g of BMA, and 0.01 g of AIBN as a radical polymerization initiator, were dissolved in 450 g of ethanol, and poured into a glass reactor vessel for polymerization. After the atmosphere was replaced with a nitrogen gas, the mixture was reacted at 50° C. for 72 hours. When the reaction completed, the reactant was purified by reprecipitation, using ethanol as a good solvent and diethyl ether as a poor solvent, and heat-dried to obtain 41 g of a polymer (referred to as P-1) in 82% yield. The composition of the starting material and the result of measurement of the molecular weight of the obtained polymer and other data are shown in Table 1.

### Synthesis Examples 1-2 to 1-4

Polymers (P-2 to P4) shown in Table 1 were synthesized by polymerization in the same way as in Synthesis Example 1-1, except that the composition of the starting material for each polymer was as shown in Table 1. The compositions of the starting materials and the results of measurement of the molecular weight of the obtained polymers and other data are shown in Table 1. As used hereinbelow, SMA is an abbreviation for stearyl methacrylate, N stands for the carbon number of the alkyl group in alkyl(meth)acrylate, and R stands for the molar fraction of alkyl(meth)acrylate.

TABLE 1

| | | Synthesis Example | | | |
|---|---|---|---|---|---|
| | | 1-1 | 1-2 | 1-3 | 1-4 |
| Code of Obtained Polymer | | P-1 | P-2 | P-3 | P-4 |
| Composition | MPC (g) | 45 | 34 | 45 | 44.5 |
| of Starting | BMA (g) | 5 | 16 | 5 | — |
| Material | SMA (g) | — | — | — | 5.5 |
| | AIBN (g) | 0.01 | 0.01 | 0.005 | 0.01 |
| | Methanol (g) | 450 | 450 | 450 | 450 |
| | Total (g) | 500 | 500 | 500 | 500 |
| | MPC/BMA | 90/10 | 68/32 | 90/10 | — |
| | (weight ratio) | | | | |
| | MPC/SMA | — | — | — | 89/11 |
| | (weight ratio) | | | | |
| Polymer | MPC/BMA | 80/20 | 50/50 | 80/20 | — |
| | (molar ratio) | | | | |
| | MPC/SMA | — | — | — | 90/10 |
| | (molar ratio) | | | | |
| | N × R | 80 | 200 | 80 | 180 |
| | Molecular Weight | 500000 | 300000 | 1000000 | 200000 |
| | Mw | | | | |
| | Mw/Mn | 3.3 | 3.9 | 4.3 | 4.2 |
| | (Dispersion | | | | |
| | Index) | | | | |

### Synthesis Examples 2-1 to 2-8

Polymers (R-1 to R-8) shown in Table 2 were synthesized by polymerization in the same way as in Synthesis Example 1-1, except that the composition of the starting material for each polymer was as shown in Table 2. The compositions of the starting materials and the results of measurement of the molecular weight of the obtained polymers and other data are shown in Table 2. As used hereinbelow, HEMA is an abbreviation for 2-hydroxyethyl methacrylate, GLMA for glycelol methacrylate, PMA for n-propyl methacrylate, MMA for methyl methacrylate, and MA for methacrylic acid.

TABLE 2

| | | Synthesis Example | | | | | | | |
|---|---|---|---|---|---|---|---|---|---|
| | | 2-1 | 2-2 | 2-3 | 2-4 | 2-5 | 2-6 | 2-7 | 2-8 |
| Code of Obtained Polymers | | R-1 | R-2 | R-3 | R-4 | R-5 | R-6 | R-7 | R-8 |
| Composition | MPC (g) | 50 | 34 | 32.5 | 30 | 45 | 25 | 47.5 | 47.5 |
| of Starting | BMA (g) | — | — | — | — | 5 | 25 | — | — |
| Material | HEMA (g) | — | 16 | — | — | — | — | — | — |
| | GLMA (g) | — | — | 17.5 | — | — | — | — | — |
| | MA (g) | — | — | — | 20 | — | — | — | — |
| | PMA (g) | — | — | — | — | — | — | 2.5 | — |
| | MMA (g) | — | — | — | — | — | — | — | 2.5 |
| | AIBN (g) | 0.01 | 0.01 | 0.01 | 0.01 | 0.05 | 0.01 | 0.01 | 0.01 |
| | Methanol (g) | 450 | 450 | 450 | 450 | 450 | 450 | 450 | 450 |
| | Total (g) | 500 | 500 | 500 | 500 | 500 | 500 | 500 | 500 |
| | MPC/BMA (weight ratio) | 100/0 | — | — | — | 90/10 | 50/50 | — | — |
| | MPC/HEMA (weight ratio) | — | 68/32 | — | — | — | — | — | — |
| | MPC/GLMA (weight ratio) | — | — | 65/35 | — | — | — | — | — |
| | MPC/MA (weight ratio) | — | — | — | 60/40 | — | — | — | — |
| | MPC/PMA (weight ratio) | — | — | — | — | — | — | 95/5 | — |
| | MPC/MMA (weight ratio) | — | — | — | — | — | — | — | 95/5 |
| Polymer | MPC/BMA (molar ratio) | 100/0 | — | — | — | 80/20 | 30/70 | — | — |
| | MPC/HEMA (molar ratio) | — | 50/50 | — | — | — | — | — | — |
| | MPC/GLMA (molar ratio) | — | — | 50/50 | — | — | — | — | — |
| | MPC/MA (molar ratio) | — | — | — | 30/70 | — | — | — | — |
| | MPC/PMA (molar ratio) | — | — | — | — | — | — | 90/10 | — |
| | MPC/MMA (molar ratio) | — | — | — | — | — | — | — | 87/13 |
| | N × R | — | — | — | — | 80 | 280 | (30) | (13) |
| | Molecular Weight | 1000000 | 500000 | 300000 | 653000 | 90000 | 100000 | 500000 | 380000 |
| | Mw | | | | | | | | |
| | Mw/Mn (Dispersion Index) | 5 | 4.9 | 4.8 | 4.2 | 4.3 | 3.9 | 4.1 | 4.5 |

### Reference Example 1-1

An aqueous solution of a contact lens lubricant, containing 1% w/v of polymer P-1 as shown in Table 1 and 0.9% w/v of sodium chloride, was prepared. With the obtained aqueous solution, the average coefficient of friction was measured according to each of the following methods, and the standard deviation was calculated. The results are shown in Table 3.

<Measurement of Average Coefficient of Friction>

100 μL each of the above aqueous solution was instilled over a polymethylmethacrylate (abbreviated as PMMA hereinbelow) substrate and a silicon rubber sheet substrate, both of 3 cm wide and 7 cm long. Using a friction feeling tester KES-SE-DC (manufactured by KATO TECH CO., LTD.), the coefficient of friction between each substrate and a silicon probe was measured three times for each sample, and the average was taken as the average coefficient of friction. Here, PMMA was used as a general model of contact lens materials, and the silicon rubber sheet was used as a model of rigid gas permeable contact lens materials and silicon hydrogel contact lens materials. The average coefficient of friction indicates lower friction in a smaller value.

Next, an HEMA hydrogel (abbreviated as HEMA gel hereinbelow) substrate of 3 cm wide, 7 cm long, and 1 mm thick was soaked in the above aqueous solution over night. Using the friction feeling tester KES-SE-DC (manufactured by KATO TECH CO., LTD.), the coefficient of friction between the resulting HEMA gel substrate and a stainless probe was measured three times for each sample, and the average was taken as the average coefficient of friction. Here, HEMA gel was used as a general model of hydrogel soft contact lens materials. The HEMA gel had been prepared as follows.

<Preparation of HEMA Gel>

A mixture of 99.45 parts by weight of HEMA, 0.5 parts by weight of ethyleneglycol dimethacrylate, and 0.05 parts by weight of AIBN was introduced into a mold made of Teflon spacers of 1 mm thick and two polyethylene terephthalate films of 5 cm wide and 10 cm long. The mixture was heated at 60° C. for 12 hours in an oven. The resulting molded resin was soaked in about 500 mL of saline, and when reached the equilibrium swelling, soaked in about 500 mL of fresh saline. This operation was repeated twice, and the resulting material was cut into a piece of 3 cm wide and 7 cm long. The moisture content of the obtained HEMA gel was about 38%.

### Reference Examples 1-2 to 1-4

Aqueous solutions of a contact lens lubricant, each containing 1% w/v of one of the polymers P-2 to P-4 as shown in Table 1 and 0.9% w/v of sodium chloride, were prepared. With each of the obtained aqueous solutions, the average coefficients of friction were measured in the same way as in Reference Example 1-1, and the standard deviations were calculated. The results are shown in Table 3.

### Comparative Reference Examples 1-1 to 1-5

Aqueous solutions of a contact lens lubricant, each containing 1% w/v of one of the polymers R-1 to R-4 and R-6 shown in Table 2 and 0.9% w/v of sodium chloride, were prepared. With each of the obtained aqueous solutions, the average coefficients of friction were measured in the same way as in Reference Example 1-1, and the standard 10 deviations were calculated. The results are shown in Table 4.

TABLE 3

| | | Reference Example | | | |
|---|---|---|---|---|---|
| | | 1-1 | 1-2 | 1-3 | 1-4 |
| Polymer Used | | P-1 | P-2 | P-3 | P-4 |
| PMMA | Average | 0.054 | 0.062 | 0.044 | 0.038 |
| Substrate | Coefficient of | | | | |
| | Friction | | | | |
| | Standard Deviation | 0.004 | 0.004 | 0.004 | 0.006 |
| Silicon | Average | 0.566 | 0.856 | 0.848 | 0.618 |
| Substrate | Coefficient of | | | | |
| | Friction | | | | |
| | Standard Deviation | 0.016 | 0.066 | 0.008 | 0.012 |
| HEMA Gel | Average | 0.580 | 0.650 | 0.362 | 0.170 |
| | Coefficient of | | | | |
| | Friction | | | | |
| | Standard Deviation | 0.071 | 0.076 | 0.079 | 0.000 |

TABLE 4

| | | Comparative Reference Example | | | | | |
|---|---|---|---|---|---|---|---|
| | | 1-1 | 1-2 | 1-3 | 1-4 | 1-5 | 1-6 |
| Polymer Used | | R-1 | R-2 | R-3 | R-4 | R-5 | — |
| PMMA | Average | 1.302 | 0.116 | 0.524 | 1.198 | 0.156 | 1.474 |
| Substrate | Coefficient of | | | | | | |
| | Friction | | | | | | |
| | Standard | 0.056 | 0.008 | 0.028 | 0.034 | 0.012 | 0.060 |
| | Deviation | | | | | | |
| Silicon | Average | 1.452 | 1.220 | 1.094 | 1.246 | 1.604 | 1.234 |
| Substrate | Coefficient of | | | | | | |
| | Friction | | | | | | |
| | Standard | 0.164 | 0.012 | 0.066 | 0.020 | 0.072 | 0.120 |
| | Deviation | | | | | | |
| HEMA Gel | Average | 2.867 | 2.862 | 1.375 | 1.393 | 0.848 | 3.493 |
| | Coefficient of | | | | | | |
| | Friction | | | | | | |
| | Standard | 0.069 | 0.928 | 0.819 | 0.052 | 0.158 | 0.197 |
| | Deviation | | | | | | |

From Tables 3 and 4, it can be seen that all the values of the average coefficients of friction are smaller in Reference Examples 1-1 to 1-4 than in Comparative Reference Examples 1-1 to 1-6, so that it is apparent that the lubricant of the present invention has a friction-reducing effect. It can also be seen that, even if the copolymers containing MPC were used in Comparative Reference Examples 1-1 to 1-5, the friction was hardly reduced when the copolymers were not copolymer (A). It is particularly noted that the results of Comparative Reference Example 1-5, wherein polymer R-6 not meeting the conditions of the present invention only in the value of N×R was used, were inferior to those of Reference Examples. It was thus demonstrated that the aqueous lubricant solutions containing copolymer (A) exhibited an excellent friction-reducing effect even when used for soft contact lenses.

### Comparative Reference Examples 1-7 to 1-11

Aqueous solutions were prepared in the same way as in Reference Example 1-1 except that 0.9% w/v of polymer P-1 was replaced with 0.9% w/v of poly-N-vinyl pyrrolidone (abbreviated as NVP, manufactured by WAKO PURE CHEMICALS INDUSTRIES, LTD., polyvinyl pyrrolidone K90), 0.9% w/v of PVA (manufactured by KURARY CO., LTD., PVA220c), 0.5% w/v of polyethylene glycol (abbreviated as PEG, manufactured by WAKO PURE CHEMICALS INDUSTRIES LTD., polyethylene glycol 2000000), 1% w/v of hydroxyethyl cellulose (abbreviated as HEC, manufactured by DAICEL CHEMICAL INDUSTRIES, LTD., SE550), or 1% w/v of hydroxypropylmethyl cellulose (abbreviated as HPMC, manufactured by SHIN-ETSU CHEMICAL CO., LTD., 65SH-4000) With each of the obtained aqueous solutions, the average coefficients of friction with respect to the PMMA and silicon substrates were measured, and the standard deviations were calculated. The results are shown in Table 5.

TABLE 5

Comparative Reference Example

| | | 1-7 | 1-8 | 1-9 | 1-10 | 1-11 |
|---|---|---|---|---|---|---|
| Polymer Used | | NVP | PVA | PEG | HEC | HPMC |
| PMMA | Average | 0.276 | 0.334 | 0.916 | 1.282 | 0.994 |
| Substrate | Coefficient of | | | | | |
| | Friction | | | | | |
| | Standard | 0.008 | 0.004 | 0.030 | 0.042 | 0.024 |
| | Deviation | | | | | |
| Silicon | Average | 1.160 | 0.278 | 0.808 | 1.288 | 0.172 |
| Substrate | Coefficient of | | | | | |
| | Friction | | | | | |
| | Standard | 0.026 | 0.056 | 0.036 | 0.060 | 0.016 |
| | Deviation | | | | | |

From Tables 3 and 5, it can be seen that most of the values of the average coefficients of friction were smaller in Reference Examples 1-1 to 1-4 than in Comparative Reference Examples 1-7 to 1-11. It was thus demonstrated that the aqueous solutions containing the lubricant used in the present invention had a superior lubricating property compared to various conventional water-soluble polymers.

### Examples 1-1 to 1-3 and Comparative Examples 1-1 to 1-7

Contact lens wetting solutions were prepared using the polymers prepared in Synthesis Examples, various buffers, inorganic chlorides, preservatives, chelating agents, and optionally various surfactants. Composition of each wetting solution is shown in Tables 6 and 7.

As used hereinbelow, PHMB is an abbreviation for polyhexamethylene biguanide, CHG for chlorhexidine gluconate, and EDTA2Na for disodium ethylenediaminetetraacetate. Pluronic F127 and Tetronic 908 are abbreviations (trademarks) for nonionic surfactants manufactured by BASF Japan Ltd.

TABLE 6

| | | Example | | | Comparative Example | | |
|---|---|---|---|---|---|---|---|
| | | 1-1 | 1-2 | 1-3 | 1-1 | 1-2 | 1-3 |
| Blending | Polymer | P-1 | P-2 | P-3 | P-4 | P-1 | P-2 |
| Composition | Amount (% w/v) | (1.0) | (1.0) | (0.5) | (1.0) | (1.0) | (1.0) |
| | Buffer | Boric Acid | Boric Acid | Monosodium | Boric Acid | Boric Acid | Boric Acid |
| | Amount (% w/v) | (0.1) | (0.1) | Phosphate | (0.1) | (0.1) | (0.1) |
| | | Borax | Borax | (0.012) | Borax | Borax | Borax |
| | | (0.01) | (0.01) | Disodium | (0.01) | (0.01) | (0.01) |
| | | | | Phosphate | | | |
| | | | | (0.355) | | | |
| | Inorganic | NaCl | NaCl | NaCl | NaCl | NaCl | NaCl |
| | Chloride | (0.9) | (0.9) | (0.8) | (0.9) | (0.9) | (0.9) |
| | Amount (% w/v) | | | | | | |
| | Preservative | PHMB | PHMB | CHG | PHMB | PHMB | PHMB |
| | Amount (% w/v) | (0.003) | (0.003) | (0.003) | (0.003) | (0.003) | (0.003) |
| | Chelating Agent | EDTA2Na | EDTA2Na | EDTA2Na | EDTA2Na | EDTA2Na | EDTA2Na |
| | Amount (% w/v) | (0.1) | (0.1) | (0.06) | (0.1) | (0.1) | (0.1) |
| | Surfactant | — | — | — | Pluronic | Pluronic | Tetronic |
| | Amount (% w/v) | | | | F127 | F127 | 908 |
| | | | | | (0.03) | (0.1) | (0.1) |

TABLE 7

| | | Comparative Example | | | |
|---|---|---|---|---|---|
| | | 1-4 | 1-5 | 1-6 | 1-7 |
| Blending | Polymer | R-5 | R-7 | R-8 | R-1 |
| Composition | Amount (% w/v) | (1.0) | (1.0) | (1.0) | (1.0) |
| | Buffer | Boric Acid | Boric Acid | Boric Acid | Monosodium |
| | Amount (% w/v) | (0.1) | (0.1) | (0.1) | Phosphate |
| | | Borax | Borax | Borax | (0.012) |
| | | (0.01) | (0.01) | (0.01) | Disodium |
| | | | | | Phosphate |
| | | | | | (0.355) |
| | Inorganic | NaCl | NaCl | NaCl | NaCl |
| | Chloride | (0.9) | (0.9) | (0.9) | (0.8) |
| | Amount (% w/v) | | | | |
| | Preservative | PHMB | PHMB | PHMB | CHG |
| | Amount (% w/v) | (0.003) | (0.003) | (0.003) | (0.003) |
| | Chelating Agent | EDTA2Na | EDTA2Na | EDTA2Na | EDTA2Na |
| | Amount (% w/v) | (0.1) | (0.1) | (0.1) | (0.06) |

Next, 100 μL of each wetting solution prepared in Examples 1-1 to 1-3 and Comparative Examples 1-1 to 1-6 was instilled over a PMMA substrate of 3 cm wide and 7 cm long, and, using the friction feeling tester KES-SE-DC (manufactured by KATO TECH CO., LTD.), the coefficient of friction between the substrate and a silicon probe was measured three times for each sample. The substrates were then ultrasonic-cleaned in 100 mL of saline for 1 minute, and the coefficient of friction was measured again. This cleansing operation was repeated four times, while the coefficient of friction was measured after each cleansing. The measured average coefficients of friction and standard deviations are shown in Tables 8 and 9. Here, the cleansing in saline was performed as simulation of removal of the wetting solution by tear.

TABLE 8

| | | | | | Comparative | | |
|---|---|---|---|---|---|---|---|
| | | Example | | | Example | | |
| | | 1-1 | 1-2 | 1-3 | 1-1 | 1-2 | 1-3 |
| Number of | Average | 0.034 | 0.055 | 0.041 | 0.052 | 0.045 | 0.045 |
| Cleansing 0 | Coefficient of | | | | | | |
| | Friction | | | | | | |
| | Standard | 0.004 | 0.006 | 0.005 | 0.006 | 0.005 | 0.005 |
| | Deviation | | | | | | |
| Number of | Average | 0.038 | 0.056 | 0.049 | 0.122 | 0.089 | 0.145 |
| Cleansing 1 | Coefficient of | | | | | | |
| | Friction | | | | | | |
| | Standard | 0.004 | 0.006 | 0.005 | 0.013 | 0.010 | 0.016 |
| | Deviation | | | | | | |
| Number of | Average | 0.040 | 0.062 | 0.065 | 0.188 | 0.105 | 0.192 |
| Cleansing 2 | Coefficient of | | | | | | |
| | Friction | | | | | | |
| | Standard | 0.010 | 0.004 | 0.004 | 0.011 | 0.006 | 0.012 |
| | Deviation | | | | | | |
| Number of | Average | 0.148 | 0.125 | 0.185 | 0.354 | 0.264 | 0.465 |
| Cleansing 3 | Coefficient of | | | | | | |
| | Friction | | | | | | |
| | Standard | 0.006 | 0.005 | 0.008 | 0.015 | 0.011 | 0.020 |
| | Deviation | | | | | | |
| Number of | Average | 0.318 | 0.355 | 0.334 | 0.744 | 0.462 | 0.980 |
| Cleansing 4 | Coefficient of | | | | | | |
| | Friction | | | | | | |
| | Standard | 0.058 | 0.021 | 0.020 | 0.045 | 0.028 | 0.059 |
| | Deviation | | | | | | |

TABLE 9

| | | Comparative Example | | |
|---|---|---|---|---|
| | | 1-4 | 1-5 | 1-6 |
| Number of | Average | 0.064 | 0.078 | 0.059 |
| Cleansing 0 | Coefficient of | | | |
| | Friction | | | |
| | Standard | 0.004 | 0.004 | 0.004 |
| | Deviation | | | |
| Number of | Average | 0.265 | 0.362 | 1.362 |
| Cleansing 1 | Coefficient of | | | |
| | Friction | | | |
| | Standard | 0.025 | 0.006 | 0.061 |
| | Deviation | | | |
| Number of | Average | 0.377 | 1.032 | 1.442 |
| Cleansing 2 | Coefficient of | | | |
| | Friction | | | |
| | Standard | 0.070 | 0.023 | 0.064 |
| | Deviation | | | |
| Number of | Average | 0.810 | 1.355 | 1.425 |
| Cleansing 3 | Coefficient of | | | |
| | Friction | | | |
| | Standard | 0.034 | 0.057 | 0.060 |
| | Deviation | | | |
| Number of | Average | 1.421 | 1.433 | 1.430 |
| Cleansing 4 | Coefficient of | | | |
| | Friction | | | |
| | Standard | 0.060 | 0.058 | 0.064 |
| | Deviation | | | |

Table 8 shows that the wetting solutions of Examples 1-1 to 1-3 kept their average coefficients of friction at a low level even after four times of cleansing, so that it was demonstrated that these wetting solutions maintain their friction-reducing effect for a prolonged period of time. Table 9 shows that even the wetting solution of Comparative Example 1-4 containing MPC copolymer R-5 of Mw90000 had an insufficient friction-reducing effect. Comparative Examples 1-5 to 1-6 showed that, even when Mw of the MPC copolymer was not less than 100000, the wetting solution had an insufficient friction-reducing effect, if the chain of the alkyl group in the (meth)acrylate constituting the copolymer is short, such as in n-propyl methacrylate or methyl methacrylate.

Comparisons between Example 1-1 and Comparative Example 1-2 and between Example 1-2 and Comparative Example 1-3 in Table 8 indicate that the wetting solutions with a surfactant could keep their friction-reducing effect for a shorter time than those without a surfactant. It is thus understood that the wetting solutions of the present invention are preferably free of surfactants.

Next, 100 μL of the wetting solution of Example 1-1 was instilled over a PMMA substrate of 3 cm wide and 7 cm long, and, using the friction feeling tester KES-SE-DC (manufactured by KATO TECH CO., LTD.), the coefficient of friction between the substrate and a silicon probe was measured three times for each sample, and the results were compared with those of the following Comparative Examples 1-8 to 1-11. The average of the measurement results was taken as the average coefficient of friction, and the standard deviation was calculated. The results are shown in Table 10.

### Comparative Examples 1-8 to 1-11

100 μL of each of the commercially available wetting solutions, “Alcon Tears Natural II” (manufactured by ALCON LABORATORIES, INC., Comparative Example 1-8), “Riki-film” (manufactured by Santen-Allergan Corp., Comparative Example 1-9), “My Tear Hard” (manufactured by SENJU PHARMACEUTICAL CO., LTD., Comparative Example 1-10), and “OPTIMUM Wetting and Rewetting Drop” (manufactured by LOBOB LABORATORIES, INC., Comparative Example 1-11), was instilled over a PMMA substrate of 3 cm wide and 7 cm long, and the coefficient of friction between each substrate and a silicon probe was measured in the same way as above. The results are shown in Table 10.

TABLE 10

| | | Example | Comparative Example | | | |
|---|---|---|---|---|---|---|
| | | 1-1 | 1-8 | 1-9 | 1-10 | 1-11 |
| PMMA | Average | 0.042 | 0.910 | 0.572 | 0.560 | 1.002 |
| Substrate | Coefficient of | | | | | |
| | Friction | | | | | |
| | Standard | 0.006 | 0.028 | 0.030 | 0.048 | 0.024 |
| | Deviation | | | | | |

Table 10 indicates that the wetting solution of Example 1-1 remarkably reduced friction even compared to commercially-available various wetting solutions of Comparative Examples 1-8 to 1-11.

Next, 100 μL of each of the wetting solutions prepared in Examples 1-1, 1-3, and Comparative Example 1-1, and 100 μL of the wetting solution prepared in Comparative Example 1-7 or saline for comparison, was instilled over a PMMA substrate of 3 cm wide and 7 cm long, and using the friction feeling tester KES-SE-DC (manufactured by KATO TECH CO., LTD.), the coefficient of friction between the substrate and a piece of swine sclerocornea was measured three times for each sample. The average of the measured values was taken as the average coefficient of friction, and the standard deviation was calculated. The results are shown in Table 11. The coefficients of friction measured using swine sclerocornea were evaluated with reference to Journal of Japan Contact Lens Society (Ikuo Iguchi et al., Vol. 36, p317, 1994).

TABLE 11

| | | | Comp. | Comp. | |
|---|---|---|---|---|---|
| | Example | Example | Example | Example | |
| Composition | 1-1 | 1-3 | 1-1 | 1-7 | Saline |
| Average | 0.031 | 0.040 | 0.043 | 0.150 | 0.215 |
| Coefficient of | | | | | |
| Friction | | | | | |
| Standard | 0.003 | 0.004 | 0.004 | 0.019 | 0.011 |
| Deviation | | | | | |

Table 11 indicates that the wetting solutions of the present invention effectively reduced friction between the swine sclerocornea and the PMMA substrate. This result implies that the present lubricants would also provide the friction-reducing effect between human sclerocornea and the hard contact lens.

### REFERENCE EXPERIMENT

The following instillation test was conducted on rabbits using the wetting solutions prepared in Example 1-1 and Comparative Example 1-7.

<Instillation Test on Rabbit>

Rigid gas permeable contact lenses (manufactured by MENICON CO., LTD.) were put on the right eyes of three New Zealand white rabbits (2.5-3.6 kg body weight, 12-15 weeks of age). After 30 minutes from the lens insertion, a few drops of an eye drop were instilled into the eyes. Subsequently, a few drops of the wetting solution were instilled every hour in total of eight times a day. After 8 hours from the lens insertion, the lenses were taken out of the rabbits, and cleaned and soaked in a cleaning and soaking solution for rigid gas permeable contact lenses (trade name “O₂ Care” manufactured by MENICON CO., LTD.) On the next morning, the lenses were rinsed with tap water, and put back on the rabbits' eyes. This cycle was repeated for 22 days. After the completion of the test, the eyes of the rabbits were histopathologically examined. The results were evaluated and indicated by 0 when no abnormality was observed, 1 when slight abnormality, 2 when moderate abnormality, and 3 with sever abnormality. The results of the evaluation of each tissue are shown in Table 12.

TABLE 12

| | | Example | | | Comp. Example | | |
|---|---|---|---|---|---|---|---|
| Wetting Solution | | 1-1 | | | 1-7 | | |
| Number of Rabbit | | 1 | 2 | 3 | 1 | 2 | 3 |
| Tissue | Cornea | 0 | 0 | 0 | 1 | 1 | 1 |
| | Conjunctiva | 0 | 0 | 0 | 2 | 2 | 1 |
| | Iris | 0 | 0 | 0 | 0 | 0 | 0 |
| | Retina | 0 | 0 | 0 | 0 | 0 | 0 |
| | Nervus Opticus | 0 | 0 | 0 | 0 | 0 | 0 |

Table 12 shows that no injuries on the cornea and conjunctiva were observed when the wetting solution of Example was used, compared to that of Comparative Example. It is believed that the injuries observed when the wetting solution of Comparative Example was used were caused by frictional contact between the contact lens and the cornea. It was thus demonstrated that the wetting solution of Example 1-1 had a friction-reducing effect between the contact lens and the cornea or conjunctiva, and was safe for ocular tissues.

What is claimed is:

1. A contact lens wetting solution consisting of 0.05 to 5% w/v of a lubricant consisting of copolymer (A) obtained by polymerizing a monomer composition comprising 2-(meth)acryloyloxyethyl phosphorylcholine represented by the formula (1) and alkyl(meth)acrylate represented by the formula (2), 0.1 to 1.5% w/v of a buffer, 0.1 to 1.5% w/v of an inorganic chloride, 0.00001 to 0.1% w/v of a preservative, 10⁻⁴ to 0.1% w/v of a chelating agent, and water, wherein said copolymer (A) has a weight average molecular weight of 100,000 to 1,000,000, and satisfies the formula 80≦N×R≦240, provided that 4≦N≦18 and 10%≦R≦60%, wherein N stands for a carbon number of an alkyl group in said monomer Y constituting copolymer (A), and R stands for a molar fraction of a unit derived from monomer Y with respect to the sum of units derived from monomers X and Y: 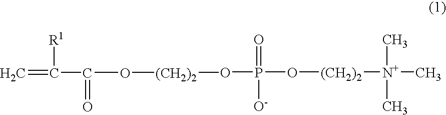
wherein R¹ stands for a hydrogen atom or a methyl group, 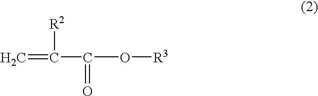
wherein R² stands for a hydrogen atom or a methyl group, and R³ stands for an alkyl group having 4 to 18 carbon atoms.

2. The wetting solution of claim 2, wherein said inorganic chloride is sodium chloride, said preservative is chlorhexidine gluconate or polyhexamethylene biguanide, and said chelating agent is disodium ethylenediaminetetraacetate.
